(19) United States
(12) Patent Application Publication
(10) Pub. No.: US 2019/0030245 A1
(43) Pub. Date: Jan. 31, 2019

(54) MANAGEMENT OF THE BLOOD GLUCOSE BALANCE OF A DIABETIC PATIENT

(71) Applicant: VOLUNTIS, Paris (FR)

(72) Inventors: Eric BAVIERE, Gazeran (FR); Geneviève D'ORSAY, Paris (FR); Guillaume MOUCHEROUD, Vanves (FR)

(21) Appl. No.: 16/071,824

(22) PCT Filed: Jan. 19, 2017
(86) PCT No.: PCT/FR2017/050108

§ 371 Date: Jul. 20, 2018

(30) Foreign Application Priority Data
Jan. 21, 2016 (FR) 16 50483

(51) Int. Cl. A61M 5/172; A61B 5/145; A61B 5/00; G16H 20/17

(52) U.S. Cl. CPC A61M 5/1723; A61B 5/14532; A61M 2205/18; G16H 20/17; A61M 2230/201; A61B 5/4839

(57) ABSTRACT

Disclosed is a medical device for managing the blood glucose balance of a diabetic patient, including: a storage module for storing a plurality of blood glucose concentrations measured over a predetermined time period, the measured blood glucose concentrations relating to a content of a blood component representing the blood glucose level of the patient; and a processing circuit that uses a management rule to detect a state of blood glucose imbalance in the patient by comparing the measured blood glucose concentrations with a range of threshold values having an upper blood glucose concentration bound corresponding, for example, to a hyperglycaemic state and a lower blood glucose concentration bound corresponding, for example, to a hypoglycaemic state, the circuit also being configured to emit a warning signal when a state of blood glucose imbalance is detected.

### TECHNICAL FIELD AND PRIOR ART

The present invention relates to the health field.

The present invention relates more particularly to software medical devices suitable for improving the therapeutic accompaniment of diabetic patients.

One of the objectives of the present invention is to design a software medical device configured for early detection of the return of the patient to a state of glycaemic imbalance in order in particular to improve the general state of health of the patient and to prevent the patient from being in a state of hyperglycaemia or hypoglycaemia for a prolonged period of time.

Diabetes is a chronic disease associated with an absence or a deficiency of insulin production; in other words, a diabetic patient cannot correctly regulate the amount of sugar in the blood.

A distinction is made between mainly two types of diabetes:
type I diabetes, or insulin-dependent diabetes (IDD), which is diabetes in which the patient's body no longer produces insulin. It applies to approximately 350 000 individuals in France;
type II diabetes, or non-insulin-dependent diabetes (NIDD), which is diabetes in which insulin production is insufficient or deficient. It results in a gradual degradation of the patient's ability to regulate their sugar level in the blood. Type II diabetes applies to more than 2 500 000 individuals in France.

In type I diabetes, insulin-based treatment, either by injection, or by pump, is the only possible therapy.

In the early stages of type II diabetes, treatment consists essentially in accompanying the patient in a modification of the latter's lifestyle acting mainly on their diet and the performing of physical activity.

In a subsequent phase, the patient then receives a first medical support, for example in the form of oral antidiabetics.

Then, as the disease and the gradual degradation of the patient's pancreatic function progress, said patient moves onto insulin injection.

This movement onto the insulin injection is an important change in the patient's life.

Fear of needles, fear of hypoglycaemia in the event of overdose, the reaction of the body to this new treatment are all parameters which make the therapeutic treatment of the patient complex.

There are numerous medical protocols allowing the accompaniment of the patient in this insulin treatment phase.

In all of these protocols, the objectives are substantially the same.

The protocols concentrate exclusively on the “titration” phase; said phase consists mainly in gradually reaching the targeted insulin dose by:
increasing, step by step, the insulin dose taken by the patient in order to prevent adverse effects associated with too rapid an increase in insulin intake. This titration phase uses, for example, a calculation algorithm based in particular on the patient's last blood glucose level, the insulin used and the last recommended dose, and the patient's insulin sensitivity;
stopping this increase as soon as the patient's blood glucose level reaches an objective predetermined by the latter's physician. This objective corresponds substantially to a blood glucose level which is in a range of values that is generally between 80 and 120 mg/dl.

Once the blood glucose level measured in the patient is in this range of values, the patient's dose is reputedly at equilibrium: the patient is consequently considered to be in a state of glycaemic balance. The term glycaemic balance phase is also used.

The applicant submits here that it is important, for medical reasons, to stop the increase in the insulin dose once the blood glucose level is in the range of expected values.

This approach has many drawbacks for the patient's health.

Indeed, the titration phase is based on the last blood glucose level measured and on the dose previously recommended for determining the change in the daily insulin dose.

Events that have taken place (such as for example physical activity, stress, forgetting the last injection, etc.) that the patient has forgotten to take into account are not considered in the calculation for dose; however, these events can directly influence the previously recommended dose.

If said previously recommended dose was over evaluated because the patient forgot to declare a hypoglycaemic event, the new dose calculation will be based on an over evaluated dose for determining the new recommendation.

Consequently, this new dose may be too high.

Such a dose (too high) may be dangerous to the patient: it may precipitate the occurrence of a further hypoglycaemia.

The applicant submits moreover that the existing dose calculations are based on the last recommended dose, and not on the last dose actually taken by the patient.

Thus, a patient having intentionally reduced their dose because of a fear of hypoglycaemia will certainly, in the next period, have a blood glucose level that is too high; the patient will consequently be recommended to increase their insulin dose, without detecting that the main cause of the imbalance at this stage is linked to the patient having taken a sub-dose.

In this case, rather than recommending a new, higher dose to the patient, the latter should be encouraged to use the dose actually recommended.

Furthermore, the reasons why a fasting blood glucose level of the patient is just below the maximum threshold may have several origins:
the patient had a reduced carbohydrate intake on the previous day (for example, the patient ate less than usual), or because
the patient consumed more sugar on the previous day (for example, by performing a particular form of physical activity).

In any event, a fasting blood glucose level of the patient may not be just below the maximum threshold because the patient reach their balance point.

However, the existing methods of adjustment do not make it possible to isolate and/or to decide not to take into account an exceptional event so as to delay reaching the insulin balance.

The consequences of this faulty interpretation regarding the interruption of the titration initiation phase are that the patient then remains imbalanced until their next visit to their physician, which may be detrimental to their health.

Finally, if any event disrupts the patient's metabolism, the patient may swing back into a state of imbalance.

With the existing methods, the patient has to wait for their next visit to the physician to re-evaluate their insulin dose; said patient may therefore remain chronically in imbalance for a long period of time.

The applicant submits that at the current time there is in the prior art no solution which makes it possible to accurately detect, early on, the appearance in a diabetic patient of a state of glycaemic imbalance during a stability phase.

### SUMMARY AND SUBJECT OF THE PRESENT INVENTION

The present invention aims to improve the current situation described above.

The present invention makes it possible to overcome the various drawbacks of the prior art mentioned above by providing a personalized management of the glycaemic balance of a diabetic patient which makes it possible in particular to detect the occurrence of a glycaemic imbalance during a phase of glycaemic balance.

To this effect, the present invention relates, according to a first aspect, to a medical device for managing the glycaemic balance of a diabetic patient.

According to the invention, the device comprises a memory module which is configured for storing a plurality of blood glucose levels measured during a balance phase over a given period of time.

Advantageously, these blood glucose levels measured are relative to a content of a blood component representative of the patient's blood glucose level.

According to the invention, the device also comprises a processing circuit implementing a management rule configured for detecting a state of glycaemic imbalance in the patient.

Preferably, the management rule provides for a comparison of the blood glucose levels measured with a threshold value range.

Preferably, this threshold value range has:
an upper blood glucose limit corresponding to a high blood glucose state (for example a fasting blood glucose level above 1.20 g/l corresponding substantially to a state of hyperglycaemia), and
a lower blood glucose limit corresponding to a low blood glucose state (for example a fasting blood glucose level below 0.80 g/l corresponding substantially to a state of hypoglycaemia).

Advantageously, the processing circuit is configured for emitting a warning signal when a state of glycaemic imbalance is detected.

Thus, by virtue of this combination of technical means, characteristic of the present invention, a medical device (for example a communication terminal) is provided which is simple to use, allowing the diabetic patient to be warned or to alert their attending physician when a state of glycaemic imbalance has been detected.

In this way, the diabetic patient, who, after a titration phase, takes their target insulin dose and thinks they are in a state of glycaemic balance, is warned of an imbalance as soon as it occurs.

They may consequently have a meeting with their attending physician so as to begin a new titration phase in order to re-achieve a state of balance.

Thus, by virtue of the present invention and of this early detection, the diabetic patient does not remain in a glycaemic state for a prolonged period of time.

In one particular embodiment, the management rule, which is implemented by the processing circuit, comprises an algorithm configured for detecting a state of glycaemic imbalance in the patient when a given percentage of blood glucose values is not within the threshold value range.

According to one possible implementation example, a state of glycaemic imbalance is detected in the patient when more than 50% of the blood glucose values are not in the threshold value range.

According to another implementation example, a state of glycaemic imbalance is detected in the patient when more than 66% of the blood glucose values are not in the threshold value range.

In another particular embodiment, the management rule, which is implemented by the processing circuit, comprises another algorithm configured for detecting a state of imbalance in the patient when the distribution of the values measured over the period of time has a standard deviation greater than a given threshold deviation.

Advantageously, the device according to the invention comprises a processor (or central circuit) configured for, on receipt of the warning signal:
deactivating the processing circuit so as to end the balance phase, and
activating a titration circuit so as to initiate a (new) titration phase making it possible to determine a new daily insulin dose.

This new titration phase then makes it possible to achieve the state of balance.

Preferably, the titration circuit is capable of implementing a dosage rule configured for calculating said new daily insulin dose as a function in particular of blood glucose values and of physiological and/or medical parameters specific to the patient.

It is understood here that the blood glucose values used to carry out this calculation are values measured during the titration phase.

These measurements may be carried out for example using a glycaemic probe.

The physiological and/or medical parameters specific to the patient are, for their part, pieces of information entered by the patient and/or by the attending physician directly or indirectly via said device, or are pieces of information resulting, for example, from measurements carried out by dedicated sensors (activity, geolocation, etc.).

The dosage rule according to the invention may also take into consideration other parameters, such as for example a target insulin dose such as that recommended by the physician during a prior consultation.

Advantageously, the dosage rule is configured:
for decreasing the daily dose by at least one unit when, during a period of at least one day, the mean of the blood glucose levels measured is strictly below the lower blood glucose limit, and
for increasing the daily dose by at least one unit when, during a period of at least one day, the mean of the blood glucose levels measured is strictly above the upper blood glucose limit.

This dosage rule integrates a computing logic in which is carried out a comparison of the mean of the blood glucose levels measured over a defined period of time relative to a given value range.

It can therefore be provided, for example, that the dosage rule varies the insulin dose proportionally to the deviation between this mean and the given value range.

Alternatively, it can be provided that the dosage rule can use steps as a function of the gap between the mean and this given value range. In this case, the number of steps and the increments specific to each step can be configured by the physician.

In any event, this dosage rule makes it possible, after detection of the state of imbalance, to initiate a new “titration” phase in order to achieve once again the glycaemic balance.

In one particular embodiment, the device comprises a control circuit configured for comparing the calculated dose with the dose injected into the patient's body.

It will be understood here that the dose referred to as dose injected into the patient's body can be the dose actually injected; this dose is provided by the injector after injection (for example by servo-control). It is for example possible to envisage the case of an injector which supplies this information to the titration circuit once the dose is injected.

Alternatively, this “injected” dose is the dose declared by the patient after injection.

Advantageously, the titration circuit is capable of communicating with the insulin injector in order to recover the information relating to the dose actually injected into the patient's body of this dose.

Advantageously, the titration circuit is capable of communicating with this insulin injector in order to transmit to the titration circuit information relating to the daily insulin dose calculated so as to inject this dose into the patient's body.

Advantageously, the device according to the present invention comprises at least one sensor with an activity configured for measuring and supplying to the processing circuit information relating to the activity of said patient.

Advantageously, the management rule is configured for processing the activity information supplied so as to detect at least one disruptive event relating to a change in activity of said patient.

Advantageously, the management rule is configured for detecting a state of glycaemic imbalance in the patient as a function of said at least one disrupting event.

In one particular embodiment, said at least one sensor comprises a movement sensor.

In another embodiment which can be combined with the preceding embodiment, said at least one sensor comprises a geolocation probe capable of supplying information on the patient's location.

Preferably, said at least one sensor is coupled to an internal clock.

This makes it possible to synchronize the data collected and measured.

Advantageously, the device according to the invention also comprises a glycaemic probe configured for measuring a content of a blood component representative of the blood glucose level of said patient.

Preferably, the glycaemic probe is configured for communicating the blood glucose levels measured to the memory module.

Advantageously, the glycaemic probe is configured for carrying out the measurements at regular time intervals, for example every day (optionally at a set time).

Preferably, the blood component is the HbA1c, or glycated haemoglobin, marker.

Advantageously, the device according to the present invention comprises display means configured for displaying the information contained in the warning signal.

Such a warning signal comprises, for example, the information relating to the state of glycaemic imbalance detected: for example, information relating to a state of hyperglycaemia or information relating to a state of hypoglycaemia.

Correspondingly, the present invention relates, according to a second aspect, to a method for managing the glycaemic balance of a diabetic patient.

According to the invention, the method is implemented by computer means and comprises the following steps:
storage of a plurality of blood glucose levels measured during a glycaemic balance phase over a given period of time, the blood glucose levels measured being relative to a content of a blood component representative of the patient's blood glucose level;
comparison of the blood glucose levels measured with a threshold value range having an upper blood glucose limit corresponding to a high blood glucose state and a lower blood glucose limit corresponding to a low blood glucose state;
detection of a state of glycaemic imbalance in the patient as a function of the implementation of a management rule analysing the results of the comparison;
in the event of detection of a state of glycaemic imbalance in the patient, emission of a warning signal.

Advantageously, during the step of analysing the results of the comparison, a state of glycaemic imbalance is detected in the patient when a given percentage of said blood glucose levels measured is not in the threshold value range.

In one particular embodiment, a state of glycaemic imbalance is detected in the patient during the analysis step when more than 50% of said blood glucose levels measured are not in said threshold value range.

In another particular embodiment, a state of glycaemic imbalance is detected in the patient during the analysis step when more than 66% of the blood glucose levels measured are not in the threshold value range.

Advantageously, during the step of analysing the comparison results, a state of glycaemic imbalance is detected in the patient when the distribution of the blood glucose levels measured over the period of time has a standard deviation of less than one given threshold deviation. This threshold deviation is preferably a function of the threshold value range.

Advantageously, the method comprises, following the receipt of a warning signal, a step of stopping the glycaemic balance phase, followed by a step of initializing the titration phase.

Preferably, the titration phase comprises as titration step during which a dosage rule is implemented in order to determine a new daily insulin dose.

In one particular embodiment, during the titration step, the dosage rule calculates the new daily insulin dose as a function of the blood glucose levels and of physiological and/or medical parameters specific to the patient.

Preferably, during the titration step, the dosage rule calculates the daily dose in the following way:
the daily dose is decreased by at least one unit when, during a period of at least one day, the mean of the blood glucose levels measured is strictly below said lower blood glucose limit, and
the daily dose is increased by at least one unit when, during a period of at least one day, the mean of the blood glucose levels measured is strictly above said upper blood glucose limit.

The method can also provide for the other cases already stated above.

Preferably, the method comprises a communication of the information relating to the daily insulin dose calculated to an insulin injector for an injection of this dose into the patient's body.

The method according to the invention advantageously comprises a first communication of the information relating to the daily insulin dose calculated to an insulin injector for an injection of this dose into the patient's body.

The method may also comprise a second communication of the information relating to the dose actually injected into the patient's body by the insulin injector.

Preferably, the method according to the present invention comprises a control step during which the dose calculated during the titration step is compared with the dose injected into the patient's body.

Specifically, it is possible for the dose injected to correspond to a dose declared by the patient. In this case, it is understood that this declared dose may be different from the dose actually injected. This makes it possible for the patient to freely choose the dose with which they inject themselves.

The embodiment proposed above therefore makes it possible to take into consideration this deviation in order to correctly recalculate the insulin dose on the next day.

Advantageously, the detection step comprises:
measurement of the patient's physical activity,
detection of at least one disrupting event relating to a change in activity of the patient.

In this case, during the analysis step, the management rule takes into consideration said at least one disrupting event for detecting a state of glycaemic imbalance in the patient.

Preferably, the method according to the invention comprises a measurement of a content of a blood component representative of the blood glucose level of the patient.

Preferably, the measurement is carried out at regular time intervals.

In one advantageous embodiment, the blood component is the HbA1c marker.

Preferably, the method according to the invention comprises displaying of the information contained in the warning signal.

The subject of the present invention also relates, according to a third aspect, to a computer program comprising instructions suitable for executing the steps of the method as described above, when said computer program is executed by at least one processor.

Such a computer program can use any programming language, and can be in the form of a source code, an object code, or an intermediate code between a source code and an object code, such as in a partially compiled form, or in any other desirable form.

Likewise, the subject of the present invention relates, according to a fourth aspect, to a computer-readable recording medium on which is recorded a computer program comprising instructions for executing the steps of the method as described above.

Of course, those skilled in the art will understand here that, the term “computer”, may be understood here as any computer device comprising a processor (or equivalent) capable of reading the instructions of the program and executing the steps of the method which are associated therewith.

It may in particular be a communication terminal of the “Smartphone” type.

It will be understood here that the computer program will allow, for example, the installation and the implementation of a software application on the communication terminal.

Firstly, the recording medium may be any entity or device capable of storing the program. For example, the medium may comprise a storage means, such as a ROM memory of microelectronic circuit type, or else a magnetic recording means or a hard disk. It may also more specifically be a memory module incorporated into the communication terminal.

Secondly, this recording medium may also be a transmissible medium such as an electrical or optical signal, such a signal possibly being conveyed via an electric or optical cable, by conventional or hertzian radio or by self-directed laser beam or by other means.

The computer program according to the invention may in particular be downloaded from an Internet-type network.

Alternatively, the recording medium may be an integrated circuit into which the computer program is incorporated, the integrated circuit [EV1] being suitable for executing the method in question or for being used in the execution of said method.

Thus, the subject of the present invention, by virtue of its various functional and structural aspects described above, makes it possible to make available to diabetic patients a medical device which allows them to be warned (directly or by means of their attending physician) of the occurrence of a glycaemic imbalance.

### BRIEF DESCRIPTION OF THE APPENDED FIGURES

Other features and advantages of the present invention will emerge from the description below, with reference to the appended FIGS. 1 and 2 which illustrate an implementation example thereof which is in no way limiting in nature and in which:

FIG. 1 represents a diagrammatic view of a device for managing the glycaemic balance of a diabetic patient according to one example of implementation of the invention;

FIG. 2 represents an organizational chart of the steps of the management method according to one example of implementation of the present invention.

### DETAILED DESCRIPTION OF THE INVENTION

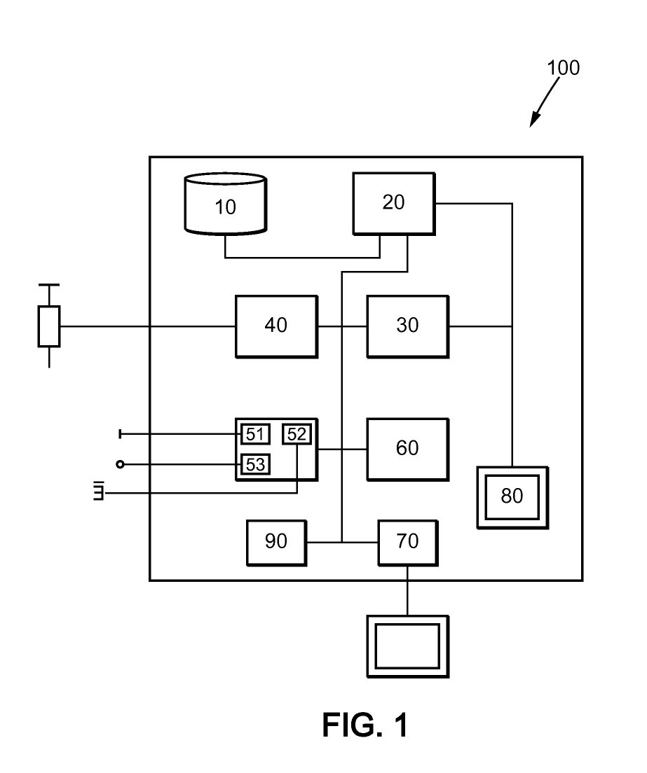

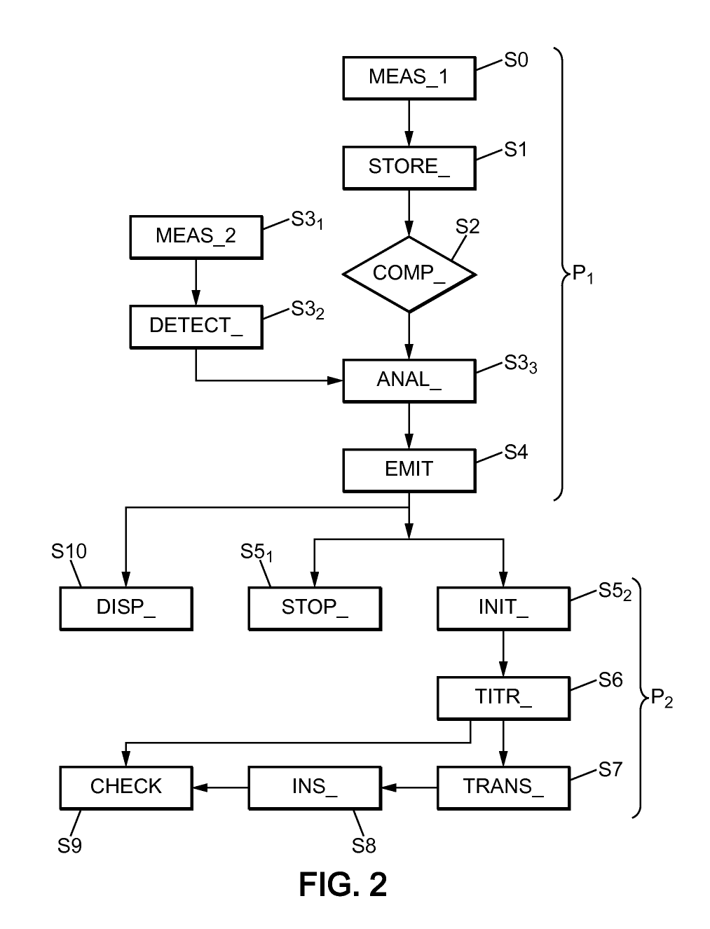

The management and the monitoring of the glycaemic state of a diabetic patient and also the system which is associated therewith will now be described in the following text with reference jointly to FIGS. 1 and 2.

As a reminder, one of the objectives of the present invention is to design a software medical device comprising computer and software means for managing and monitoring the glycaemic balance of a type II diabetic patient in order in particular to warn the patient and/or the attending physician in the event of an occurrence of a glycaemic imbalance.

This is made possible by virtue of the present invention which will be described in the text which follows according to one particular implementation example.

In this example, the diabetic patient goes to their attending physician who determines, according to the patient's medical and/or physiological parameters, a target blood glucose level to be achieved.

The physician then prescribes said patient with a medical device 100.

Such a device 100 is provided for ensuring the monitoring and the accompaniment of the patient in their treatment.

More particularly, in this example, this device 100 is a communication terminal on which is installed a dedicated medical application using software functionalities for the accompaniment of the patient.

The term SMBG [for Self-Monitoring of Blood Glucose] device is also used.

It is known that self-monitoring of blood glucose in a patient suffering from type II diabetes is important.

This self-monitoring must fall within an approach of education of the patient and of their entourage.

When an SMBG device 100 is prescribed, it is essential to explain to the patient the challenges of their treatment and to organize this self-monitoring with said patient: frequency, setting of times, glycaemic objectives, modifications of the treatment to be carried out by the patient or the physician as a function of the results.

It is known moreover that the patient must have a suitable diet and suitable physical activity.

This example represents the case where the patient has reached their “balance” insulin dose; this case represents the situation in a glycaemic balance phase, denoted P1.

This phase P1 therefore follows a prior titration.

As is very often the case, this balance phase P1 is not always long-lasting: diabetes is progressive and unstable by nature. The treatment must therefore be re-evaluated regularly in terms of all its components.

As a reminder, one of the objectives of the present invention is the early detection of the occurrence of a glycaemic imbalance.

To this effect, the device 100 comprises, in this example, a glycaemic probe 53 which measures, during a step S0, a content of a blood component representative of the patient's blood glucose level.

Preferably, the measurement relates to the HbA1c marker.

Indeed, most of the drug strategies for treating type II diabetes have retained this marker for effectively controlling the patient's blood glucose level.

In the case of the HbA1c marker, the HbA1c target of less than or equal to 7% is recommended. The drug treatment should be instituted or re-evaluated if the HbA1c is greater than 7%.

Other markers may be measured and analysed.

Preferably, this measurement S0 is done periodically, for example every day or every week.

In the example described here, the glycaemic probe 53 then communicates the blood glucose value(s) measured to a memory module 10.

This memory module, (optionally) integrated into the device 100, stores these values.

Preferably, the device 100 comprises an internal clock 60 which makes it possible to time-stamp the blood glucose levels measured and to make sure that all the information stored in the memory module 10 are synchronized with one another.

In this example, the device 100 then comprises a processing circuit 20 for processing the blood glucose levels measured.

More specifically, this circuit 20 implements a management rule which will detect a glycaemic imbalance state in the patient by carrying out a comparison S2 of each of the blood glucose levels measured and stored on the memory module 10 with a given threshold value range.

Preferably, this value range has an upper blood glucose limit corresponding substantially to a hyperglycaemia state and a lower blood glucose limit corresponding substantially to a hypoglycaemia state.

More particularly, the management rule implemented on the processing circuit 20 carries out an analysis S3₃ of the results of the comparison.

In this example, the management rule comprises an algorithm which will detect a glycaemic imbalance state in the patient when more than 66% of the blood glucose values measured are not in the threshold value range.

In this example, it is therefore understood that the management rule implemented on the circuit 20 makes it possible to analyse the distribution of the values over time.

A distribution which exhibits high variations will therefore reveal the occurrence of an imbalance state. Such an analysis makes it possible to avoid incorrect detection that would be due for example to a disrupting event.

In one particular embodiment, sensors are also provided, such as for example a movement sensor 51 or a sensor 52 of GPS type making it possible to locate the patient.

The information supplied by these sensors 51 and 52 may thus be processed and taken into consideration by the management rule for detecting the occurrence of an imbalance state.

Indeed, intense physical activity, stress, jet lag, isolated fatigue may directly influence the patient's blood glucose.

The management rule thus integrates the taking of these parameters into account in its algorithm in order to avoid an incorrect detection.

When an imbalance is detected, the processing circuit 20 generates and emits, for the intention of the processor 80, a warning signal during a step S4.

This signal comprises in particular information regarding the imbalance: for example a hypoglycaemia or hyperglycaemia state.

Optionally, this information may be displayed during a step S10 on a screen 70 of the device 100.

In the example described here, in the event of an imbalance being detected, the processor 80 thus receives the warning signal.

On receipt of this signal, it deactivates, during a step S5₁, the processing circuit 20.

This stopping step S5₁ thus brings to an end the glycaemic balance phase P1.

The processor 80 then activates, during an initializing step S5₂, a titration circuit 30. This step S5₂ makes it possible to trigger the titration phase P2.

This phase P2 aims mainly to recalculate a new daily insulin dose so as to achieve once again glycaemic balance.

The titration circuit 30 thus comprises a dosage rule which performs this calculation during a titration step S6 as a function in particular of blood glucose values and of physiological and/or medical parameters specific to said patient.

It will be understood here that the blood glucose measurements may be carried out by the glycaemic probe 40 and that the physiological and/or medical parameters specific to the patient are pieces of information stored beforehand in the memory module 10 subsequent, for example, to a first visit to the attending physician.

During this titration step S6, the dosage rule makes it possible to:
decrease said daily dose by at least one unit when, during a period of at least one day, the mean of the blood glucose levels measured is strictly below said lower blood glucose limit, and
increase said daily dose by at least one unit when, during a period of at least one day, the mean of the blood glucose levels measured is strictly above said upper blood glucose limit.

Each time that the daily insulin dose is calculated by the titration circuit 30, this information is sent, during a step S7, to an injector 70.

Wireless communication means may for example be used.

The injector 70 thus receives this information, and the patient can carry out their insulin injection during a step S8.

In the example described, a control circuit 80 is provided which will receive, from the injector 70, the information relating to the dose actually injected.

This circuit 80 will thus be able to compare this dose with the theoretical dose calculated by the titration circuit 30.

This control S9 will allow the titration circuit 30 to subsequently readjust the insulin dose(s) calculated in the subsequent titrations.

It is in fact possible for the dose actually injected to be slightly different from the calculated dose.

In the example described here, the titration circuit 30 carries out an analysis of the blood glucose levels measured during this titration phase.

This analysis then makes it possible to detect that the glycaemic balance is once again achieved: for example, the dose has been stabilized for several days and the blood glucose data are predominantly in the expected range.

In this case, the circuit 30 is provided for emitting, in turn, a balance signal.

On receipt of this signal, the processor 80 will deactivate the titration circuit 30 and will reactivate the processing circuit 20.

A repeating method which is orchestrated and managed by the processor 80 and which makes it possible to swing from a balance phase to a titration phase as a function of the patient's glycaemic state (balance or imbalance) is thus obtained.

It will be noted that the dosage and management rules differ in particular in that the titration circuit carries out its observation and its analysis of the blood glucose values over a relatively short period of time, whereas the processing circuit and the step of identifying the balance in the titration circuit are based on longer periods.

Those skilled in the art will understand here that the objective of this approach is to decrease the sensitivity of the mechanism during the balance phase; reference is also made to amortization.

Blood glucose is in fact a factor that is by nature physiologically unstable. The management of this balance phase proposed in the context of the present invention makes it possible to curb the risks associated with oscillations of this instability.

By detecting upstream the occurrence of a glycaemic imbalance in a patient, the present invention anticipates the intervention of the physician by making it possible to swing, without waiting, the patient back into a titration phase so as to dynamically calculate a new insulin dose.

Preferentially, the swing can be executed in the other direction when, for example, the balance is once again achieved.

Such an SMBG system makes it possible to dynamically manage the glycaemic state of a patient and prevents the occurrence of glycaemic accidents in the diabetic patient.

Finally, it will be possible to provide an embodiment (not represented here) with a safety circuit which makes it possible to trigger an immediate decrease in the dose if the blood glucose is below a certain threshold corresponding to hypoglycaemia.

This safety circuit can deactivate the other circuits and send a warning to the physician. Only the physician will then be able to reactivate one or other of the circuits.

It shall be observed that this detailed description relates to a particular example of implementation of the present invention, but that this description is in no way of nature limiting the subject of the invention; quite the contrary, it has the objective of removing any possible inaccuracy or any incorrect interpretation of the claims which follow.

It shall also be observed that the reference signs between parentheses in the claims which follow are not in any way limiting in nature; the only aim of these signs is to improve the intelligibility and the understanding of the claims which follow and also the scope of the protection sought.

1-32. (canceled)

33. A medical device for managing the glycaemic balance of a diabetic patient, said device comprising:
a memory module configured for storing a plurality of blood glucose levels measured during a glycaemic balance phase over a given period of time, said blood glucose levels measured being relative to a content of a blood component representative of the blood glucose level of said patient, and
a processing circuit implementing a management rule configured for detecting a glycaemic imbalance state in the patient by comparing said blood glucose levels measured with a threshold value range having an upper blood glucose limit corresponding to a high blood glucose state and a lower blood glucose limit corresponding to a low blood glucose state,
said processing circuit being configured for emitting a warning signal when a glycaemic imbalance state is detected.

34. The medical device of claim 33, in which said management rule comprises an algorithm configured for detecting a glycaemic imbalance state in the patient when a given percentage of said blood glucose levels measured is not in said threshold value range.

35. The medical device of claim 33, in which the management rule comprises an algorithm configured for detecting a glycaemic imbalance state in the patient when the distribution of said blood glucose levels measured over said period of time has a standard deviation above a given threshold deviation.

36. The medical device of claim 33, comprising a processor configured for, on receipt of the warning signal:
deactivating said processing circuit so as to bring an end to the balance phase, and
activating a titration circuit so as to initiate a titration phase for determining a new daily insulin dose.

37. The medical device of claim 36, in which the titration circuit is capable of implementing a dosage rule configured for calculating said new daily insulin dose as a function in particular of blood glucose values and of physiological and/or medical parameters specific to said patient.

38. The medical device of claim 37, in which said dosage rule is configured for:
decreasing said daily dose by at least one unit when, during a period of at least one day, the mean of the blood glucose levels measured is strictly below said lower blood glucose limit, and
increasing said daily dose by at least one unit when, during a period of at least one day, the mean of the blood glucose levels measured is strictly above said upper blood glucose limit.

39. The medical device of claim 38, comprising a control circuit configured for comparing said calculated dose with the dose injected into the body of said patient.

40. The medical device of claim 38, in which said titration circuit is capable of communicating with an insulin injector in order to recover the information relating to the dose actually injected into the body of said patient by said insulin injector.

41. The medical device of claim 39, in which said titration circuit is capable of communicating with said insulin injector in order to transmit to said injector the information relating to said calculated dose so as to inject this said dose into the body of said patient.

42. The medical device of claim 33, comprising at least one activity sensor configured for measuring and supplying information relating to the activity of said patient.

43. The medical device of claim 42, in which said management rule is configured for:
processing said activity information so as to detect at least one disrupting event relating to a change in activity of said patient, and
detecting a glycaemic imbalance state in the patient as a function of said at least one disrupting event.

44. The medical device of claim 42, in which said at least one sensor comprises a movement sensor.

45. The medical device of claim 42, in which said at least one sensor comprises a geolocation probe capable of supplying information relating to the location of said patient.

46. The medical device of claim 42, in which said at least one sensor is coupled to an internal clock.

47. The medical device of claim 33, comprising a glycaemic probe configured for measuring a content of a blood component representative of the blood glucose level of said patient.

48. The medical device of claim 47, in which said glycaemic probe is configured for carrying out said measurement at regular time intervals.

49. A method for managing the glycaemic balance of a diabetic patient, said method carried out by computer means comprising the following steps:
storage of a plurality of blood glucose levels measured during a glycaemic balance phase over a given period of time, said blood glucose levels measured being relative to a content of a blood component representative of the blood glucose level of said patient;
comparison of said blood glucose levels measured with a threshold value range having an upper blood glucose limit corresponding to a high blood glucose state and a lower blood glucose level corresponding to a low blood glucose state;
detection of a glycaemic imbalance state in the patient as a function of the implementation of a management rule analysing the results of said comparison;
in the event of detection of an imbalance state in the patient, emission of a warning signal.

50. The method of claim 49, in which, during the analysis step, a glycaemic imbalance state is detected in the patient when a given percentage of said blood glucose values is not in said threshold value range.

51. The method of claim 50, in which, during the analysis step, a glycaemic imbalance state is detected in the patient when more than 50% of said blood glucose values are not in said threshold value range.

52. The method of claim 50, in which, during the analysis step, a glycaemic imbalance state is detected in the patient when more than 66% of said blood glucose values are not in said threshold value range.

53. The method of claim 49, comprising, following the receipt of a warning signal, a step of stopping the glycaemic balance phase, followed by a step of initializing the titration phase, said titration phase comprising a titration step during which a dosage rule is implemented for determining a new daily insulin dose.

54. The method of claim 53, in which, during the titration step, the dosage rule calculates said new daily insulin dose as a function of blood glucose values and of physiological and/or medical parameters specific to said patient.

55. The method of claim 54, comprising a second communication of the information relating to said dose actually injected into the body of said patient by said insulin injector.

56. The method of claim 54, comprising a control step during which said dose calculated during the titration step is compared with the dose injected into the body of the patient.

57. The method of claim 49, in which the detection step comprises:
measurement of the physical activity of said patient,
detection of at least one disrupting event relating to a change in activity of said patient,
in which, during the analysis step, said management rule takes into consideration said at least one disrupting event for detecting a glycaemic imbalance state in the patient.

58. A non-transitory computer-readable recording medium on which is recorded a computer program comprising instructions which, when executed by a computer, perform the steps of the method according to claim 49.
